(19) United States
(12) Patent Application Publication
(10) Pub. No.: US 2004/0082557 A1
(43) Pub. Date: Apr. 29, 2004

(54) Methods for treating estrogen-dependent disorders

(76) Inventors: Charles Paul Wajszczuk, (US); Hendrik J. DeKoning Gans, Tokyo (JP); Enrico Di Salle, Milan (IT); Gabriella Piscitelli, Milan (IT); Giorgio Massimini, Milan (IT); Dinesh Purandare, Bryn Mawr, PA (US)

Correspondence Address: Arent Fox Kintner Plotkin & Kahn, PLLC, Suite 600, 1050 Connecticut Ave., N.W., Washington, DC, 20036, US

(21) Appl. No.: 10/611,653

(22) Filed: Jul. 2, 2003

Related U.S. Application Data
(63) Continuation-in-part of application No. 09/770,911, filed on Jan. 26, 2001, now abandoned.
(63) Continuation-in-part of application No. PCT/EP02/00638, filed on Jan. 18, 2002.
(60) Provisional application No. 60/393,320, filed on Jul. 2, 2002.

(51) Int. Cl. A61K 31/56; A61K 31/135

(52) U.S. Cl. 514/182; 514/646

(57) ABSTRACT

A method of preventing and/or treating estrogen-dependent disorders selected from endometriosis, uterine fibroids, dysfunctional uterine bleeding, endometrial hyperplasia, polycystic ovarian disease, fibrocystic breast disease and fibrocystic mastopathy, is disclosed which is comprised of administering to a female mammal in need of such treatment an effective amount of aromatase inactivator exemestane, alone or in combination with additional therapeutic agents. The present invention also relates to a method for treating infertility in a female mammal in need of the infertility treatment, comprising administering an effective amount of exemestane to the mammal.

### CROSS-REFERENCE TO RELATED APPLICATIONS

This is a continuation-in-part of PCT/EP02/00638 filed Jan. 18, 2002, which claimed the priority of U.S. application Ser. No. 09/770,911 filed Jan. 26, 2001, and claims the benefit of U.S. Provisional Application No. 60/393,320 filed Jul. 2, 2002. The disclosures of application Ser. Nos. 09/770,911, PCT/EP02/00638 and 60/393,320 are incorporated by reference.

### FIELD OF THE INVENTION

The present invention relates to methods of preventing and/or treating hormone-dependent disorders, in particular, estrogen-dependent disorders selected from endometriosis, uterine fibroids, dysfunctional uterine bleeding, endometrial hyperplasia, polycystic ovarian disease, benign breast disease and fibrocystic mastopathy, which comprises administering to a patient in need thereof the aromatase inactivator exemestane, alone or in combination with additional therapeutic agents. The invention also relates to a method of treating infertility in a female mammal in need of infertility treatment, comprising administering an effective amount of exemestane to the mammal.

### BACKGROUND OF THE INVENTION

Endometriosis is a disease in which patches of endometrial tissue, which normally is found only in the uterine lining (endometrium), grow outside the uterus. The misplaced endometrial tissue commonly adheres to the ovaries and the ligaments that support the uterus as well as the peritoneal lining of the abdominal cavity. Because the misplaced endometrial tissue responds to the same hormones that the uterus responds to, it may bleed during the menstrual period, often causing cramps, pain, irritation, and the formation of scar tissue. Moreover, it has been demonstrated that endometriotic tissue expresses aromatase activity, not seen in normal endometrium.

Considerable circumstantial and laboratory evidence suggests that endometriosis is an estrogen-dependent disease. The main source for circulating estrogens in the premenopausal women is the ovary, where androgens are converted to estrogens by the enzyme aromatase. It has been assumed that estrogens are delivered to endometriotic implants via circulation. However, it has been recently demonstrated that significant levels of aromatase activity and mRNA are also present in the stromal component of the endometriotic tissue, whereas aromatase expression was either absent or barely detectable in the eutopic endometrium. In addition, prostaglandin (PG)E2, which is present in very high levels in endometriotic tissues, was found to be the most potent inducer of aromatase activity in endometriosis-derived stromal cells. The production of PGE2 in endometrial stromal cells, in turn, was demonstrated to be simulated by cytokines and estradiol via enhancement of cyclooxygenase-2 (COX-2) expression, the enzyme responsible for the synthesis of PGE2. Therefore, aberrant regulation of the aromatase enzyme in endometriotic tissues, which favor increased local level of estradiol, is possibly involved in the development and growth of endometriosis.

In general, the aims of treatment of a patient with endometriosis include elimination of the misplaced endometriotic tissue, relief of pain and induction of pregnancy. Current treatments include administration of drugs that suppress the activity of the ovaries and slow the growth of endometrial tissue, surgery to remove the misplaced endometriotic tissue, surgical removal or the uterus, fallopian tubes and/or ovaries, or combinations of those treatments. While drug treatments are less invasive than surgery, administration of drugs such as combination estrogen-progestin oral contraceptives, progestins, danazol, and gonadotropin-releasing hormone (GnRH) agonists (such as Buserelin) is accompanied by multiple unwanted side-effects associated with hormone modulation, including bleeding between periods, hot flushes, predisposition to osteoporosis and mood swings. Furthermore, as yet available drug treatment doesn't cure endometriosis; the disease usually returns after treatment is stopped.

Benign breast disease, or often called fibrocystic breast disease, appears to be dependent on ovarian steroids. See Jacquemier et al., Cancer, 49, 2534 (1982). Aromatase inhibitors have not been tried in this disease, but antiestrogens seem to be of benefit. See Ricciardi & Ianniruberto, Obstet. Gynecol., 54, 80 (1979).

Uterine fibroids, which appear in the reproductive years and regress after menopause, are the result of cellular proliferation and differentiation in the uterine tissue regulated by the ovarian steroids. At present, treatment of a patient suffering from uterine fibroids include surgery and administration of GnRH agonists.

Fibrocystic mastopathy is a condition considered in the past to confer an increased risk for breast cancer. Reevaluation of the outcome of this disorder has concluded that the overall increased risk of 1.86 of developing breast cancer, estimated by pooling together many published series, was more likely due to the selection of patients than to the real malignant potential of the disease. The presence of proliferation with cell atypia on pathologic assessment, however, is associated with an increased risk for breast cancer, especially if the patient has a positive family history. Fibrocystic disease occurs more often among individuals 30 to 55 years of age, and is frequently identified by women as multiple, round lumps in one or both breasts. The mammographic patterns of multiple areas of fibrosis and cysts are typical, but represent a difficult background for evaluation of an underlying neoplasia.

Human infertility is defined in Harrison Dictionary as the inability to conceive after 12 months of unprotected sexual intercourse. There is a spectrum of infertility, ranging from reduced conception rates or the need of medical intervention to irreversible causes of infertility. Infertility can be attributed primarily to male factors in 25%, female factors in 58%, and is unexplained in about 17% of couples.

Ovulation is the process where an ovum or ova are released from the ovaries. The timing of ovulation within the menstrual cycle is of foremost importance for fertilization. It is well known that follicles acquire the ability to ovulate following growth and maturation stimulated by the pituitary gonadotropins. Ovulation induction is a therapeutic procedure commonly used to manage infertile patients. Ovulation induction is employed in particular for the following two purposes: 1) to treat anovulation in patients with hypogonadotropic hypogonadism, polycystic ovarian disease and other menstrual cycle disorders and 2) to stimulate multiple folliculogenesis in patients (mostly with normal menstrual cycles) who are candidates for assisted reproduction techniques. These procedures are also termed controlled ovarian stimulation or hyperstimulation. However there are several complications caused by ovulation induction, including for instance multiple gestations and ovarian hyperstimulation syndrome. The complications mostly occur in polycystic ovary syndrome patients and/or full-dose gonadotropin regimens.

Polycystic ovarian disease is one of the most common causes of infertility in women. The disease appears to result from an abnormality in steroid metabolism, and the current major form of therapy in this disease is the antiestrogen, clomiphene. See Yen, Clin. Endocrinol., 12, 177 (1980). Endometriosis is estimated to occur in about 10 to 15 percent of menstruating women between the ages of 25 to 44. As many as 25 to 50 percent of infertile women may have endometriosis, which can physically interfere with conception.

Exemestane is 6-methylenandrost-1,4-diene-3,17-dione, disclosed in U.S. Pat. No. 4,808,616. Exemestane is endowed with a peculiar mechanism of aromatase inhibition. The aromatase enzyme (450ₐᵣₒₘ) is a specific form of cytochrome P450 hemoprotein composed of a P450 (heme) moiety and a peptidic moiety. The enzyme catalyzes a multistep reaction leading to aromatization of the A ring of the androgen substrate (mainly androstenedione) to estrone, requiring the presence of the cofactor NADPH. After this enzymatic reaction, the enzyme molecule is once more available to perform a new aromatization. The exemestane's mechanism of aromatase inhibition has been extensively studied and the compound has been found to cause enzyme inactivation. In fact exemestane, structurally related to the natural substrate androstenedione, is initially recognized by the aromatase enzyme as a false substrate, therefore it competes with androstenedione at the active site of the enzyme. The compound is then transformed (through a NADPH-dependent mechanism) to an intermediate which binds irreversibly to the enzyme causing its inactivation (also known as suicide inhibition). Therefore the enzyme is definitely inactivated and de novo enzyme synthesis is required for estrogen production.

### SUMMARY OF THE INVENTION

One of the objects of the present invention is directed to a method of preventing and/or treating an estrogen-dependent disorder selected from endometriosis, uterine fibroids, dysfunctional uterine bleeding, endometrial hyperplasia, polycystic ovarian disease, fibrocystic breast disease and fibrocystic mastopathy, which method comprises administering to a female mammal in need of such treatment an effective amount of exemestane, alone or in combination with one or more additional therapeutic agents. The present invention also includes a method of treating infertility in a female mammal in need of the treatment, comprising administering an effective amount of exemestane to the mammal.

### DETAILED DESCRIPTION OF THE INVENTION

The present invention relates to a method of preventing and/or treating an estrogen-dependent disorder selected from endometriosis, uterine fibroids, dysfunctional uterine bleeding, endometrial hyperplasia, polycystic ovarian disease, fibrocystic breast disease and fibrocystic mastopathy, which comprises administering to a female mammal in need of such treatment an effective amount of exemestane, either alone or in combination with at least one additional therapeutic agent, thus achieving a therapeutic effect. When exemestane is used in combination with at least one additional therapeutic agent, exemestane and the at least one therapeutic agent can be administered simultaneously, separately or sequentially to the mammalian patient in amounts sufficient to achieve a therapeutically useful effect and being sufficiently close in time to achieve the therapeutically useful effect.

Within the scope of the present invention is a method of treating infertility in a female mammal in need of the treatment, comprising administering an effective amount of exemestane to the mammal. The “effective amount” is an amount therapeutically effective in treating fertility. The “effective amount” can be a therapeutically effective follicular stimulating amount of exemestane. Ovarian follicular stimulation refers to a process in which exemestane is used to bring about ovulation in a female mammal lacking ovulation, wherein induction of follicular rupture and ovulation of fertilizable oocytes are produced. As used herein, the term “a therapeutically effective follicular stimulating amount” refers to an amount which is effective, upon single or multiple dose administration to the female mammal, in treating infertility, e.g. by inducing ovarian follicular stimulation either when being taken or after the administration is stopped causing a rebound hyperstimulation of the ovaries.

The infertility being treated according to the present invention, preferably, is anovulatory infertility. Infertility caused by or associated with hypogonadotropic hypogonadism can also be treated by the method of the present invention with the administration of an effective amount of exemestane. Also preferably, the infertility treated by the method of the invention is infertility caused by or associated with an estrogen-dependent disorder. For instance, the method of the present invention is effective in treating infertility caused by or associated with endometriosis or polycystic ovarian disease.

According to a further preferred embodiment of the invention, a method is provided for inducing ovarian follicular stimulation in a female mammal suffering from hypogonadotropic hypogonadism, polycystic ovarian disease or other menstrual cycle disorders, or who is a candidate for assisted reproduction techniques, comprising administering a therapeutically effective follicular stimulating amount of exemestane to the mammal. The effect of exemestane on ovarian follicular stimulation can for instance be seen in animal models once its administration is stopped with a resultant increase in follicle development and rupture.

As used herein, the terms “polycystic ovarian disease” and “polycystic ovary syndrome” are interchangeable and have the same meaning as polycystic ovarian syndrome (PCOS).

Examples of “estrogen-dependent disorder” include endometriosis, uterine fibroids, dysfunctional uterine bleeding, endometrial hyperplasia, polycystic ovarian disease, fibrocystic breast disease, fibrocystic mastopathy and infertility.

In this patent application, the term “female mammal” includes, for example, humans, horses, bovines, dogs and cats. The female mammal is preferably a female human. In the method of treating infertility, preferred examples of such female mammal are patients with hypogonadotropic hypogonadism, polycystic ovary syndrome and other menstrual cycle disorders, and patients who otherwise are candidate for assisted reproduction techniques.

The present invention also provides the use of exemestane in the manufacture of a medicament for preventing and/or controlling an estrogen-dependent disorder selected from endometriosis, uterine fibroids, dysfunctional uterine bleeding, endometrial hyperplasia, polycystic ovarian disease, fibrocystic breast disease and fibrocystic mastopathy, in a patient undergoing a simultaneous, separate or sequential treatment with another therapeutic agent.

A further object of the invention is the use of exemestane in the manufacture of a medicament for use in treating infertility in a female mammal. The invention also provides the use of exemestane in the manufacture of a medicament for use in inducing ovarian follicular stimulation in a female mammal.

The invention also provides a product containing exemestane and at least one additional therapeutic agent as a combined preparation for simultaneous, separate or sequential administration in preventing and/or controlling an estrogen-dependent disorder selected from endometriosis, uterine fibroids, dysfunctional uterine bleeding, endometrial hyperplasia, polycystic ovarian disease, fibrocystic breast disease and fibrocystic mastopathy.

The combination preparation according to the invention can also include combination packs or compositions in which the constituents are placed side by side and can be administered simultaneously, separately of sequentially to one and the same human being. Accordingly, exemestane and the at least one additional therapeutic agent may be present within a single or distinct containers.

Accordingly, the invention also provides kits or single packages containing the pharmaceutical compositions useful for the combination treatment of the estrogen-dependent disorder discussed above. The kits or packages may also contain instructions to use the pharmaceutical compositions in accordance with the present invention.

The prevention and/or control of the above mentioned estrogen-dependent disorders by combined administration of a therapeutically effective amount of exemestane and a therapeutically effective amount of the at least one additional therapeutic agent can produce a therapeutic effect which is greater than that obtainable by single administration of the therapeutically effective amount of either exemestane solely or the at least one additional therapeutic agent alone. Such combined therapy provides a synergistic or super-additive therapeutic effect. Most importantly, the therapeutic effect is not paralleled by the toxic effects, otherwise caused by single administration of either therapeutically effective amounts of exemestane or of the at least one additional therapeutic agent.

The at least one additional therapeutic agent for combination therapy with exemestane is for instance an agent selected from danazol, a COX-2 inhibitor, a non-steroidal anti-inflammatory compound (NSAID), a retinoid compound, a matrix metallo-protease inhibitor, an anti-estrogen, GnRH agonist or antagonist, a selective progestin receptor modulator (SPRM) and an angiogenesis inhibitor, or a mixture thereof.

The combination preparation of the invention can comprise exemestane and one or more, preferably 2, 3 or 4, in particular 2, additional therapeutic agents selected from danazol, a COX-2 inhibitor, a non-steroidal anti-inflammatory compound (NSAID), a retinoid compound, a matrix metallo-protease inhibitor, an anti-estrogen, GnRH agonist or antagonist, a selective progestin receptor modulator (SPRM) and an angiogenesis inhibitor, or a mixture thereof.

Danazol, an androgen derivative which suppresses the pituitary-ovarian axis by inhibiting the release of GnRH, is well known in the art.

A COX-2 inhibitor is for instance a compound according to claims 34 to 41 of WO 00/38730. These compound are as follows:

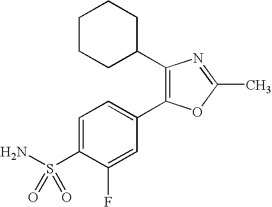

JTE-522 (4-(4-cyclohexyl 2-methyloxazol-5-yl)-2-fluorobenzenesulfonamide), 5-chloro-3-(4-(methylsulfonyl)phenyl)-2-(Methyl-5-pyridinyl) pyridine, 2-(3,5-difluorophenyl)-3-4(methylsulfonyl)phenyl)-2-cyclopenten-1-one,

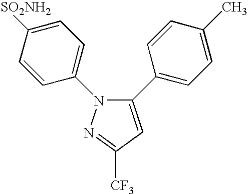

4-[5-(4-methylphenyl)-3-(trifluoromethyl)-1H-pyrazol-1-yl]-benzenesulfonamide,

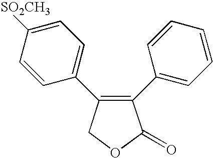

rofecoxib, (4-(4-(methylsulfonyl)phenyl]-3-phenyl-2(5H)-furanone),

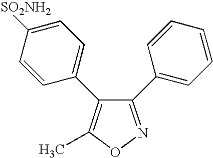

4-(5-methyl-3-phenylisoxazol-4-yl) benzenesulfonamide,

N-[[4-(5-methyl-3-phenylisoxazol-4 μl phenyl]sulfonyl]propanamide,

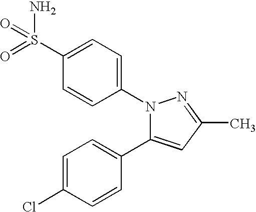

4-[5-(4-chorophenyl)-3-(trifluoromethyl)-1H-pyrazole-1-yl]benzenesulfonamide,

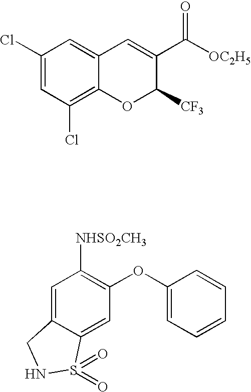

N-(2,3-dihydro-1,1-dioxido-6-phenoxy-1,2-benzisothiazol-5-yl) methanesulfonamide,

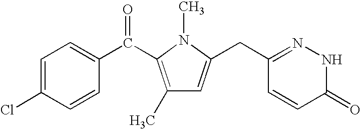

6-[[5-(4-chlorobenzoyl)-1,4-dimethyl-1H-pyrrol-2-yl]methyl]-3(2H)-pyridazinone,

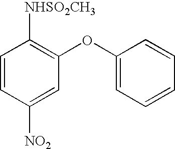

N-(4-nitro-2-phenoxyphenyl) methanesulfonamide,

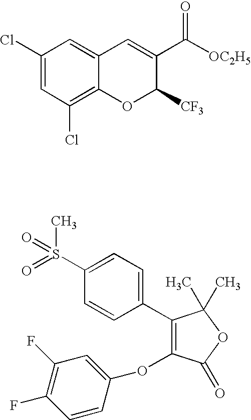

3-(3,4-difluorophenoxy)-5,5-dimethyl-4-[4-(methylsulfonyl) phenyl]-2 (5H)-furanone,

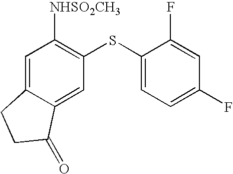

N-[6-[(2,4-difluorophenyl) thio]-2,3-dihydro-1-oxo-1H-inden-5-yl]methanesulfonamide,

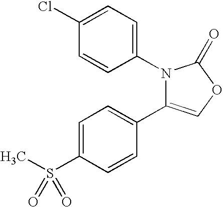

3-(4-chlorophenyl)-4-[4-(methylsulfonyl)phenyl]-2 (3H)-oxazolone,

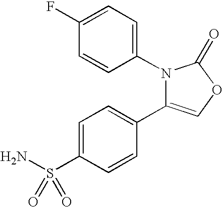

4-[3-(4-fluorophenyl)-2,3-dihydro-2-oxo-4-oxazolyl]benzenesulfonamide,

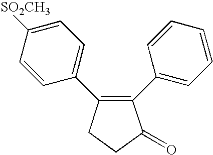

3[4-(methylsulfonyl) phenyl]-2-phenyl-2-cyclopenten-1-one,

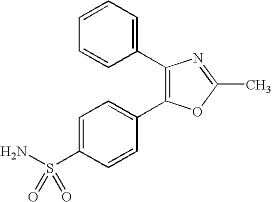

4-(2-methyl-4-phenyl-5-oxazolyl) benzenesulfonamide,

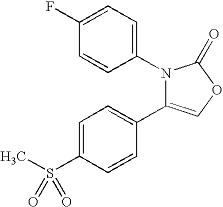

3-(4-fluorophenyl)-4-[4-(methylsulfonyl) phenyl]-2 (3H)-oxazolone,

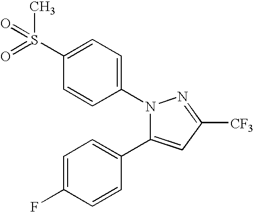

5-(4-fluorophenyl)-1-[4-(methylsulfonyl) phenyl-3-(trifluoromethyl)-1H-pyrazole,

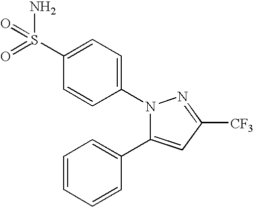

4-[5-phenyl)-3-(trifluoromethyl)-1H-pyrazol-1-yl) benzenesulfonamide,

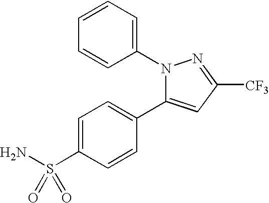

4-[1−phenyl-3-(trifluoromethyl)-1H-pyrazol-5-yl]benzenesulfonamide,

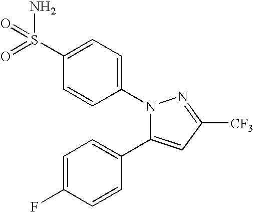

4-[5-(4-fluorophenyl)-3-(trifluoromethyl)-1H-pyrazol-1-yl]benzenesulfonamide,

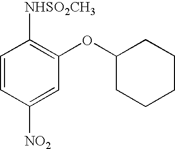

N-[2-(cyclohexyloxy)-4-nitrophenyl]methanesulfonamide,

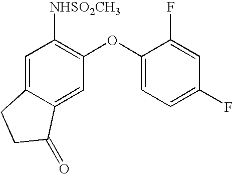

N-[6-(2,4-difluorophenoxy)-2,3-dihydro-1-oxo-1H-inden-5-yl]methanesulfonamide,

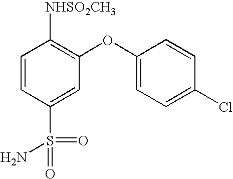

3-(4-chlorophenoxy)-4-[(methylsulfonyl) amino]benzenesulfonamide,

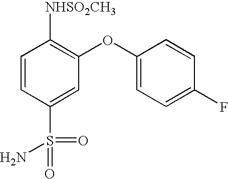

3-(4-fluorophenoxy)-4-[(methylsulfonyl) amino]benzenesulfonamide,

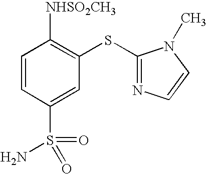

3-[(1-methyl-1H-imidzaol-2-yl)thio]-4 [(methylsulfonyl) amino]benzenesulfonamide,

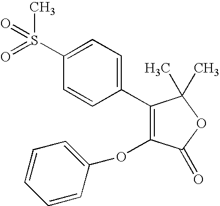

5,5-dimethyl-4-[4-(methylsulfonyl) phenyl]-3-phenoxy-2(5H)-furanone,

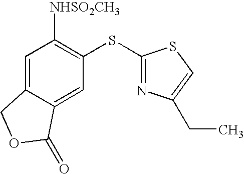

N-[6-[(4-ethyl-2-thiazolyl)thio]-1,3-dihydro-1-oxo-5-isobenzofuranyl]methanesulfonamide,

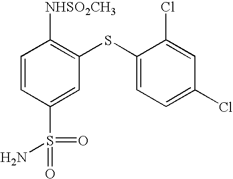

3-[(2,4-dichlorophenyl)thio]-4-[(methylsulfonyl) amino]benzenesulfonamide,

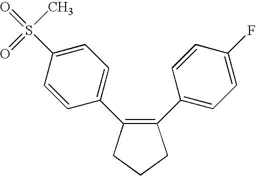

1-fluoro-4-[2-[4-(methylsulfonyl) phenyl]cyclopenten-1-yl]benzene,

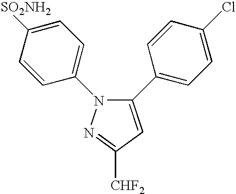

4-[5-(4-chlorophenyl)-3-(difluoromethyl)-1H-pyrazol-1-yl]benzenesulfonamide,

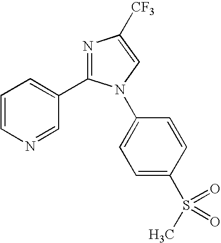

3-[1−[4-(methylsulfonyl) phenyl]-4-(trifluoromethyl)-1H-imidazol-2-yl]pyridine,

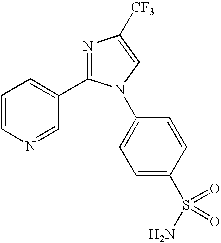

4-[2-(3-pyridinyll)-4-(trifluoromethyl)-1H-imidazol-1-yl]benzenesulfonamide,

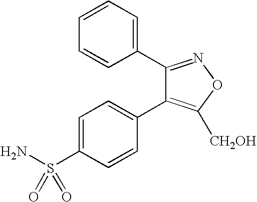

4-[5-(hydroxymethyl)-3-phenylisoxazol-4-yl]benzenesulfonamide,

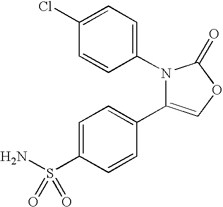

4-[3-(4-chlorophenyl)-2,3-dihydro-2-oxo-4-oxazolyl]benzenesulfonamide,

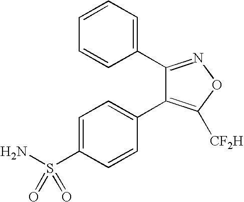

4-[5-(difluoromethyl)-3-phenylisoxazol-4-yl]benzenesulfonamide,

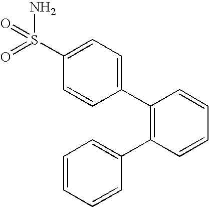

[1,1′:2′,1″-terphenyl]-4-sulfonamide,

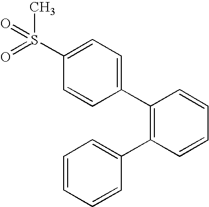

4-(methylsulfonyl)-1,1′, 2], 1″-terpheynyl,

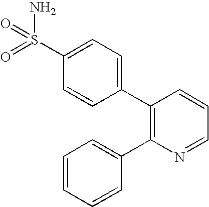

4-(2-phenyl-3-pyridinyl) benzenesulfonamide,

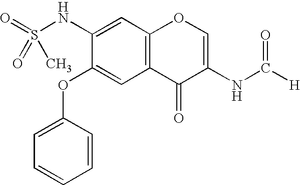

N-[3-(formylamino)-4-oxo-6-phenoxy-4H-1-benzopyran-7-yl]methanesulfonamide,

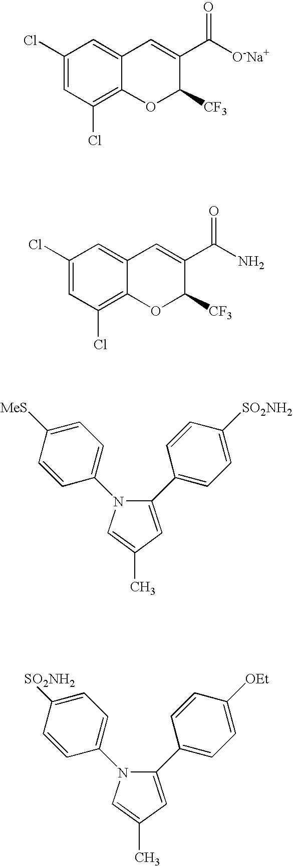

Preferred examples of COX-2 inhibitors are compound T 614 (Toyama), darbufelone (Parke-Davis), compound L745337 (Merck Frosst), celecoxib, compound CT3 (Channel Terapeutics), rofecoxib, compound L783003 (Merck & Co.), compound JT3 522 (Japan Tobacco), compound 754 (Phytochemindo Reska), parecoxib, compound S2474 (Shianogi), compound LAS 3381-5 (Almirall-Prodesfarma), valdecoxib and compound MK 663 (Merck & Co.). More preferably celecoxib, rofecoxib, parecoxib and valdecoxib, in particular celecoxib.

A non-steroidal anti-inflammatory compound (NSAID), according to the invention, is e.g. a compound selected from acetyl salicylic acid, indometacin, sulindac, phenylbutazone, diclofenac, fentiazac, ketorolac, piroxicam, tenoxicam, mecoxicam, meloxicam, cinnoxicam, ibufenac, ibuprofen, naproxen, ketoprofen, nabumetone, niflumic acid and nimesulide, or a pharmaceutically acceptable salt thereof. Preferred NSAIDs are diclofenac, piroxicam, tenoxicam, mecoxicam, meloxicam, ibufenac, ibuprofen, naproxen and ketoprofen, or a pharmaceutically acceptable salt thereof.

Examples of retinoid compounds according to the invention include, for example, Accutane; Adapalene; Allergan AGN-193174; Allergan AGN-193676; Allergan AGN-193836; Allergan AGN-193109; Aronex AR-623; BMS-181162; Galderma CD-437; Eisai ER-34617; Etrinate; Fenretinide; Ligand LGD-1550; lexacalcitol; Maxia Pharmaceuticals MX-781; mofarotene; Molecular Design MDI-101; Molecular Design MDI-301; Molecular Design MDI-403; Motretinide; Eisai 4-(2-[5-(4-methyl-7-ethylbenzofuran-2-yl)pyrrolyl])benzoic acid; Johnson & Johnson N-[4-[2-thyl-1-(1H-imidazol-1-yl)butyl]phenyl]-2-benzothiazolamine; Soriatane; Roche SR-11262; Tocoretinate; Advanced Polymer Systems trans-retinoic acid; UAB Research Foundation UAB-8; Tazorac; TopiCare; Taiho TAC-101, and Vesanoid.

Examples of matrix metallo-protease inhibitors according to the invention include known:

1-cyclopropyl-N-hydroxy-4-[[4-[4-(trifluoromethoxy)phenoxy]phenyl]sulfonyl]-4-piperidinecarboxamide monohydrochloride;

N-hydroxy-1-(phenylmethyl)-4-[[4-[4-(trifluoromethoxy)phenoxy]-1-piperidinyl]sulfonyl]-4-piperidinecarboxamide monohydrochloride;

N-hydroxy-1-(pyrdinylmethyl)-4-[[4-[4-(trifluoromethyl)phenoxy]phenyl]sulfonyl]-4-piperidinecarboxamide dihydrochloride;

N-hydroxy-2,3-dimethoxy-6-[[4-[4-(trifluoromethyl)phenoxy]-1-piperidinyl]sulfonyl]benzamide;

N-hydroxy-1-(4-pyridinylmethyl)-4-[[4-[4-(trifluoromethyl)phenoxy]phenyl]sulfonyl]-4-piperidinecarboxaimide dihydrochloride;

N-hydroxy-1-(3-pyridinylmethyl)-4-[[4-[4-(trifluoromethyl)phenoxy]phenyl]sulfonyl]-4-piperidinecarboxamide dihydrochloride;

N-hydroxy-1-(2-pyridinylmethyl)-4-[[4-[4-(trifluoromethyl)phenoxy]phenyl]sulfonyl]-4-piperidinecarboxamide monohydrochloride;

British Biotech BB-2516 (marimastat), N4-[2,2-dimethyl-1-[(methylamino)carbonyl]-propyl]-N1,2-dihydroxy-3-(2-methylpropyl)-, [2S-[N4(R*), 2R*, 3S*]]-);

BMS 275291 disclosed in WO 97/19075;

Bayer Ag Bay-12-9566 (tanomastat), 4-[(4′-chloro[1,1-diphenyl]-4-yl)oxy]-2[-(phenylthio)methyl]butanoic acid;

Agouron Pharmaceuticals AG-3340, N-hydroxy-2,2′-dimethyl-4-[[4-(4-pyridinyloxy)phenyl]sulfonyl]-3-thiomorpholinecarboxamide;

CollaGenex Pharmaceuticals CMT-3 (metastat), 6-demethyl-6-deoxy-4-dedimethylaminotetracycline, batimastat (BB-94); and

Chiroscience D-2163, 2-[1S-([(2R,S)-acetylmercapto-5 phthalimido]pentanoyl-L-leucyl)amino-3-methylbutyl]imidazole.

An anti-estrogen, e.g. a selective estrogen receptor modulator (SERM), is preferably a SERM devoid of uterotrophic activity. Examples of SERMs, according to the invention, are tamoxifen, toremifene, arzoxifene, idoxifene, EM 800, fulvestrant and droloxifene.

Examples of GnRH (LHRH) agonists according to the invention are, e.g., leuprorelin, deslorelin, triptorelin, buserelin, nafarelin, goserelin, avorelin, histerelin, compound PTL 03001 (5-oxo-L-propyl-L-histidyl-L-tryptophyl-L-seryl-L-tyrosyl-D-tryptophyl-L-leucyl-L-arginyl-N-ethyl-L-prolinamide) (Peptech), compound AN 207 (6-[N6-[5-[2-[1,2,3,4,6,11-hexahydro-2,5,12-trihydroxy-7-mehoxy-6,11-dioxo-4-[[2,3,6-trideoxy-3-(2,3-dihydro-1 H-pyrrol-1-yl).alpha.-L-lyxo-hexopyranosyl]oxy]-2-naphthacenyl]-1,5-dioxopentyl]-D-lysine]-, (2S-cis)-) (ASTA Medica Inc.), compound AN 238 L-threoninamide, N-[5-[2-[(2S,4S)-1,2,3,4,6,11-hexahydro-2,5,12-trihydroxy-7-methoxy-6,11-dioxo-4-[[2,3,6-trideoxy-3-(2,3-dihydro-1H-pyrrl-1-yl).alpha.-L-lyxo-hexopyranosyl]oxy]-2-naphthacenyl]-2-oxoethoxy]-1,5-dioxopentyl]-D-phenylalanyl-L-cysteinyl-L-tosyl-D-tryptophyl-L-lysyl-L-valyl-L-cysteinyl-cyclic (2.fwdarw.7)-disulfide (ASTA Medica Inc.) and compound SPD 424 (LHRH-hydrogel implant) (Shire Pharmaceuticals Group), or a pharmaceutically acceptable salt thereof. Preferred examples are triptorelin, leuprorelin and goserelin, or a pharmaceutically acceptable salt thereof, in particular triptorelin or a pharmaceutically acceptable salt thereof, e.g. as triptorelin pamoate.

Examples of GnRH (LHRH) antagonists, according to the invention are e.g. cetrorelix, abarelix, ramorelix, teverelix, ganirelix, compounds A 75998 (Acetyl-D-(2-naphthyl)alanyl-D-(4-chlorophenyl)alanyl-D-(3-pyridyl)alanyl-seryl-(N-methyl)tyrosyl-N-6-(nicotinoyl)-D-lysyl-leucyl-N-6-(isopropyl)lysyl-propyl-D-alaninamide) and A 84861 (Tetrahydrofuran-2-(S)-ylcarbonyl-glycyl-D-(2-napthyl)alanyl-D-(4-cholro)phenylalanyl-D-(3-pyridyl)-alanyl-L-(N-methyl)tyrosyl-D-[N6-(3-pyridylcarbonyl)]lysyl-L-leucyl-L-(N-6-isopropyl)lysyl-L-propyl-D-alanylamide) (Abbot Labs.), GnRH immunogen (Aphton Co.), compound T 98475 (Isopropyl 3-(N-benzyl-N-methylaminomethyl)-7-(2,6-difluorobenzyl)-4,7-dihydro-2-(4-isobutyrylaminophenyl)-4-oxothieno[2,3-bpyridine-5-carboxylate hydrochloride) (Takeda), and compound MI 1544 (Acetyl-D-tryptophyl-D-cyclopropyl-alanyl-D-tryptophyl-L-seryl-tyrosyl-D-lysyl-L-leucyl-L-arginyl-L-propyl-D-alaninamide), or a pharmaceutically acceptable salt thereof. Preferred example is abarelix or a pharmaceutically acceptable salt thereof.

Examples of selective progestin receptor modulators (SPRMs), according to the invention, are e.g. dienogest or a pharmaceutically acceptable salt thereof.

An angiogenesis inhibitor is e.g. an αvβ3 integrin inhibitor, a protein kinase inhibitor, angiostatin, platelet factor 4 (endostatin), a VEGF inhibitor or thalidomide.

Vascular endothelial growth factor (VEGF) inhibitors and telomerase inhibitors are well known in the art. For instance, compounds SU 5416 and SU 6668, cited herein, are also VEGF inhibitors.

Moreover known VEGF inhibitors or antagonists are agents which suppress angiogenesis by reducing binding of VEGF to cellular receptors, including but not limited to, for example blocking monoclonal antibodies against the growth factor (e.g. rhuMAbVEGF, Ryan et al., Toxicol Pathol 1999, 27:78-86), against the receptor (e.g. DC101 and derivatives, Witte et al., Cancer Metastasis Rev 1998, 17:155-61), soluble forms of VEGF receptors (e.g. soluble Flt, Aiello et al., Proc Natl Acad Sci USA 1995, 92:10457-61), or compounds which directly antagonise interactions between VEGF and cell surface receptors (e.g. Fairbrother et al., Biochemistry 1998, 37:17754-64).

A protein kinase inhibitor, according to the invention, is for instance a tyrosine kinase inhibitor, in particular compound SU6668, i.e. 3-[4-(2-carboxyethyl-3,5-dimethylpyrrol-2-yl)methylidenyl]-2-indolinone, and compound SU5416, i.e. 3-[(2,4-dimethylpyrrol-5-yl)methylidenyl]-2-indolinone, which are known from WO 96/40116 and WO 99/61422.

Examples of αvβ3 integrin inhibitors are known:

Vitaxin antibody (Ixsys); Merck KgaA EMD-121974, cyclo[RGDF-N(Me)V-];

(10S)-10,11-dihydro-3-3-(2-pyridinylamino)propoxy]-5H-dibenzo[a,d]cycloheptene-10-acetic acid;

(2S)-7-[[(1H-benzimidazol-2-ylmethyl)methylamino]carbonyl]-2,3,4,5-tetrahydro-4-methyl-3-oxo-1H-1,4-benzodiazepine-2-acetic acid;

(2S)-2,3,4,5-tetrahydro-4-methyl-7-[[[(5-methyl-1H-imidazo[4,5-b]pydin-2-yl]methyl]amino]carbonyl]-3-oxo-1H-1,4-benzodiazepine-2-acetic acid;

(bR)-b-[[[(3R)-2-oxo-3-[2-(5,6,7,8-tetrahydro-[1,8]-naphthyridin-2-yl)ethyl]1-1-pyrrolidinyl]acetyl]amino]-d-(1H-indol-3-yl)pentanoic acid; and

(3R)-N-[3-hydroxy-5-[(1,4,5,6-tetrahydro-5-hydroxy-2-pyrimidinyl)amino]benzoyl]-glycyl-3-(3-bromo-5-chloro-2-hydroxyphenyl)-b-alanine (compound SD 7784).

Angiostatin, endostatin and thalidomide are well known in the art. Pharmaceutically acceptable salts of the compound mentioned herein are well known in the art.

In effecting treatment of a patient in a therapy/prophylactic method according to the invention, exemestane and the other therapeutic agent can be administered in any form or mode which makes the compounds bioavailable in effective amounts, including oral and parenteral routes. Exemestane can be administered in any form or mode, which makes the compound bioavailable in therapeutically effective amounts. For example, routes of administration include oral, sublingual, intranasal, subcutaneous, intradermal, intraperitoneal, intramuscularly, intravenous, transdermal, vaginal, rectal and the like. Oral or intramuscular administration is generally preferred. One skilled in the art of preparing formulations can readily select the proper form and mode of administration depending upon the particular circumstances. For instance, examples of suitable oral forms are tablets, capsules, sugar and film coated tablets.

By the term a method for “controlling” and “treating” an estrogen-dependent disorder, as used herein, it is meant a method of achieving a therapeutically useful effect, which can include curing such disorder. The term “therapeutically useful effect”, besides curing such disorder, also means giving relief from symptoms, such as discomfort or pain, accompanying such disorder, in particular in patients suffering from endometriosis.

Accordingly the invention also provides a method for improving the endometriosis pain symptoms of dismenorrea, dyspareunia and pelvic pain, in a patient suffering from endometriosis comprising administering to said patient exemestane alone or with another therapeutic agent, in amounts and being close in time sufficient to achieve a therapeutically useful effect.

The term “close in time” means that in the combined method of treatment according to the invention, exemestane may be administered simultaneously with a further therapeutic agent or the compounds may be administered sequentially, in either order, to achieve a therapeutic effect.

By the term “administered” or “administering” as used herein is meant any acceptable manner of administering a drug to a patient which is medically acceptable including parenteral and oral administration.

By “parenteral” is meant intravenous, subcutaneous, intra-nasal, pulmonary, intradermal or intramuscular administration.

Oral administration includes administering exemestane of the constituents of the combined preparation in a suitable oral form such as, e.g., tablets, capsules, suspensions, solutions, emulsions, powders, syrups and the like.

The actual preferred method and order of administration of the combined preparations of the invention may vary according to, inter alia, the particular pharmaceutical formulation of exemestane being utilized, the particular pharmaceutical formulation of the other therapeutic being utilized, the particular estrogen-dependent disorder to be prevented or treated and the particular patient being treated.

In the combined method of prevention or treatment according to the subject invention, exemestane may be administered simultaneously with the other therapeutic agent or the compounds may be administered sequentially, in either order. Preferably the compounds are administered sequentially. In particular when the combination treatment comprises exemestane and a GnRH agonist or antagonist, preferably, the compounds are administered in such a way that in the patient both inhibition of hormone output of her ovaries and inhibition/inactivation of aromatase enzyme are contemporaneously provided, and thus a therapeutic useful effect is achieved.

The dosage ranges for the administration of the combined preparation may vary with the age, condition and extent of the disease in the patient and can be determined by one of skill in the art.

The dosage regimen must therefore be tailored to the particular of the patient's conditions, response and associate treatments in a manner which is conventional for any therapy, and may need to be adjusted in response to changes in conditions and/or in light of other clinical conditions.

According to the method of preventing and treating estrogen-dependent disorders in mammals, provided the present invention, exemestane for instance can be administered orally in a dosage range varying from about 2.5 mg daily to about 600 mg daily, in particular from about 10 to about 50, more preferably from about 10 to about 25 mg daily, or parenterally in a dosage ranging from about 50 to about 500 mg per injection.

Exemestane can be administered to a woman, for instance orally, at a dosage range varying from about 5 mg/day to about 200 mg/day, possibly in divided doses, e.g. 2, 3 or 4 divided doses.

According to a preferred schedule of treatment of infertility, exemestane is administered in the early part of the menstrual cycle (day 5 to day 7) and then stopped or it is administered throughout the entire cycle and then discontinued, in order to achieve the desired effective hematic follicular stimulating hormone level.

As a preferred embodiment of prevention and/or treatment of estrogen-dependent disorders, exemestane may be orally administered in the form of a complex with cyclodextrins, in particular exemestane/β-cyclodextrin complex, at a daily dosage ranging from about 10 to about 20 mg, preferably about 15 or 20 mg.

The effective therapeutic amounts of the other therapeutic agents to be used in combination with exemestane, according to the invention, are in general those commonly used in therapy for such compounds. More specifically, a therapeutically effective amount of another therapeutic agent means an amount of a compound, which when administered in combination with exemestane, is effective to prevent or treat estrogen dependent disorders, as herein defined.

Determination of a therapeutically effective amount is well within the capability of those skilled in the art. For instance an effective amount of compound SU 5416 or SU 6668 is an amount in accordance with the teaching of WO 99/61422.

An effective amount of compound SD 7784 is from about 10 to about 300 mg/kg, preferably per os, in particular from about 20 to about 200 mg/kg.

An effective amount of thalidomide may be in the range of about 1100 to about 400 mg/day.

An anti-estrogen can be administered in a dosage according to the common practice, e.g. in a dosage of about 0.1 to about 30 mg/Kg body weight per day.

An effective amount of tamoxifen may be in the range of about 10 to about 40 mg/day. An effective amount of fulvestrant may be in the range of about 50 mg to about 300 mg/day i.m., in particular of about 100 to about 250 mg/day i.m.

An effective amount of raloxifen may be in the range of about 5 to about 350 mg/day, in particular about 60 mg/day.

An effective amount of a COX-2 inhibitor may be in the range of about 0.1 to about 2000 mg, preferably in the range of about 0.5 to about 500 and most preferably between about 1 and about 200 mg. In particular as to celecoxib, rofecoxib, parecoxib and valdecoxib, a daily dosage of about 0.01 to about 100 mg/Kg body weight, preferably between about 0.1 and about 50 mg/Kg body weight may be appropriate. The daily dosage can be administered in one to four doses per day.

More particularly, as to celecoxib a dosage from about 50 to about 500 mg, in particular about 200 mg, once or twice a day may be appropriate.

As to rofecoxib the dosage normally ranges from about 12.5 to about 50 mg/day. The route of administration is preferably systemic e.g. oral or parenteral, in particular intravenous or intramuscularly.

Therapeutic dosages for SPRMs range between 2 to 50 mg/day.

Therapeutic dosages for GnRH agonists/antagonists like leuprolide are administered i.m. in doses varying from 1.5 to 15 mg, preferrably around 3.75 mg per month or 112.75 mg per 3 months.

Goserelin can be administered as goserelin acetate by subcutaneous administration of slow release goserelin at a dosage from about 3 to about 12 mg.

Triptorelin can be administered for instance as triptorelin pamaote by intramuscular administration of a sustained release formulation, in such a way that there is an interval from about 1 to 4 months between each administration and at a dosage from about 3 to about 20 mg. In particular triptorelin pamoate can be administered intramuscularly in the form of microparticles as described in U.S. Pat. No. 5,225,205 and U.S. Pat. No. 5,776,885, and more specifically as 1-month depot formulation 3.75 mg.

An effective amount of a NSAID, according to the invention is generally the one commonly used in therapy for such compound. For instance an effective amount of naproxen may be in the range of about 300 mg to about 750 mg once or twice a day.

An effective amount of piroxicam may be in the range of about 15 mg to about 50 mg once or twice a day.

An effective amount of acetyl salicylic acid may be in the range of about 150 to about 1000 mg once or twice a day.

According to a preferred feature of the invention it is here provided a method of treating and preventing an estrogen-dependent disorder selected from endometriosis, uterine fibroids, dysfunctional uterine bleeding, endometrial hyperplasia, polycystic ovarian disease, fibrocystic breast disease and fibrocystic mastopathy, in a mammal in need of such treatment, including humans, comprising administering simultaneously, separately or sequentially to said mammal exemestane and a COX-2 inhibitor selected from celecoxib, rofecoxib, parecoxib and valdecoxib, in particular celecoxib and rofecoxib, especially celecoxib, in amounts and close in time sufficient to produce a therapeutically useful effect.

According to a further preferred feature of the invention it is herein provided the use of exemestane in the manufacture of a medicament for preventing or controlling an estrogen-dependent disorder selected from endometriosis, uterine fibroids, dysfunctional uterine bleeding, endometrial hyperplasia, polycystic ovarian disease, fibrocystic breast disease and fibrocystic mastopathy, in a patient undergoing a simultaneous, separate or sequential treatment with a COX-2 inhibitor selected from celecoxib, rofecoxib, parecoxib and valdecoxib, in particular celecoxib and rofecoxib, especially celecoxib.

In a preferred embodiment of the combination therapy according to the invention, exemestane is administered orally at about 25 mg/day and celecoxib is administered orally at a dosage of about 200 mg, one or twice a day.

According to a preferred feature of the invention it is here provided a method of treating and/or preventing an estrogen-dependent disorder selected from endometriosis, uterine fibroids, dysfunctional uterine bleeding, endometrial hyperplasia, polycystic ovarian disease, fibrocystic breast disease and fibrocystic mastopathy, in a mammal in need of such treatment, including humans, comprising administering simultaneously, separately or sequentially to said mammal exemestane and a GnRH agonist or antagonist selected from triptorelin, cetrorelix, and leuprolide, in particular triptorelin and leuprolide, in amounts and close in time sufficient to produce a therapeutically useful effect.

According to such preferred features exemestane is administered orally at about 25 mg/day; triptorelin and leuprolide one or every three months at a dose of about 3 or about 20 mg, respectively, in particular of about 3.75 or about 12.75 mg, respectively.

According to a further preferred feature of the invention it is here provided the use of exemestane in the manufacture of a medicament for preventing or controlling an estrogen-dependent disorder selected from endometriosis, uterine fibroids, dysfunctional uterine bleeding, endometrial hyperplasia, polycystic ovarian disease, fibrocystic breast disease and fibrocystic mastopathy, in a patient undergoing a simultaneous, separate or sequential treatment with a GnRH agonist or antagonist selected from triptorelin, cetrorelix, and leuprolide, in particular triptorelin and leuprolide more preferably triptorelin.

As an example a kit according to the present invention provides an exemestane 25 mg oral or 50-500 mg parenteral composition and a triptorelin depot formulation 3.75 mg.

A pharmaceutically composition containing exemestane and/or another therapeutic agent according to the invention can be prepared according to well known techniques to those skilled in the art.

A pharmaceutical composition for intramuscular administration containing triptorelin pamoate in the form of a depot formulation can be prepared for instance as described in U.S. Pat. No. 5,225,205 and U.S. Pat. No. 5,776,885.

A pharmaceutical composition containing exemestane can be prepared according to U.S. Pat. No. 4,808,616. In particular an exemestane/β-cyclodextrin complex formulation can be obtained as follows:

Exemestane 20 mg Tablet

| | | |
|---|---|---|
| Composition: | exemestane | 20.00 mg |
| | Beta-cyclodextrin | 178.00 mg |
| | Avicel PH 101 | 75.00 mg |
| | Explotab | 24.00 mg |
| | Magnesium stearate | 3.00 mg |

According to methods well known in the art an exemestane/cyclodextrin kneaded system can be prepared.

All references cited in this disclosure are incorporated herein by reference.

Some of the aspects of the present invention are demonstrated with working examples below. However, the working examples are for illustration purposes only and the scope of the present invention should not be limited by the working examples.

### EXAMPLE 1

The therapeutic effect of exemestane, either alone or in combination with an additional therapeutic agent, according to the invention, was shown in an animal model of endometriosis in adult female rats. Endometriosis was induced by autotransplantation of a section of endometrium to a site under the renal capsule in adult female rats. In non-treated rats, the endometrial transplants grew progressively during the following 4 weeks. The effect of the aromatase inhibitor exemestane and the GnRH agonist triptorelin on the growth of the endometrial explants was studied by giving the compound alone or in combination for 4 weeks. Either exemestane, given intramuscularly once a week for 4 consecutive weeks, or triptorelin, given subcutaneously once weekly for 4 weeks, caused a dose-related decrease in the volume of the explant, measured in animals laparotomized one week after the fourth weekly drug dose. When the compounds were given in combination at marginally or intermediate effective doses, an additive or synergistic effect was observed.

### EXAMPLE 2

A randomized, double-blind, parallel group clinical study was performed to evaluate the effect of exemestane on the treatment of pain symptoms in premenopausal women between the age of 18 and 40 years who were

(a) recently diagnosed with endometriosis confirmed by laparoscopy or other surgical procedure without any surgical treatment; or

(b) having recurrent symptoms associated with endometriosis for at least 3 months with endometriosis having been diagnosed laparoscopically within the past 36 months (if a woman subject had laparoscopy with surgical treatment, she must be 3 months post laparoscopy at screen).

To participate in this clinical study, the woman subjects also had to meet the following requirements:

(1) having symptomatic endometriosis with a minimal total pain score equal to or greater than 4 (minimum score of 2 was required for both dysmenorrhea and pelvic pain; a woman subject could take part in the study without dyspareunia) out of a total score of 9 as the combined score on the Endometriosis Symptom Severity (ESS) scale;

(2) having regular menstrual periods (within intervals between 21 and 36 days) during the past 3 months;

(3) with a normal pap smear at the screen or within the last 12 months;

(4) not pregnant as confirmed by a urine pregnancy test at each visit during the study;

(5) not breast feeding;

(6) not having a history of or active hepatic disease (defined as a plasma concentration of aspartate aminotransferase, alanine aminotransferase or total bilirubin greater than 2 times above the upper limit of normal) or renal disease (defined as having creatinine greater than 1.5 mg/L);

(7) not using hormonal agents, including oral contraceptives, gonadotropin-releasing hormone agonists such as clomiphene citrate or Dandazol, or glucocorticoids, or any drug at doses that would suppress the hypothalamic-pituitary adrenal axis within the past 3 months prior to the start of the screen period or at any time during the study;

(8) not using anticoagulant within the past month;

(9) having a history of malignancies, other than basal cell carcinoma;

(10) having been diagnosed with anemia (Hgb of 9:0 g/dl or below);

(11) having any significant disease or any clinically significant laboratory abnormalities that, in the opinion of the investigator, might jeopardize the woman subject's health or well-being through participation in the study;

(12) not having any condition making compliance with the study instructions unlikely;

(13) known hypersensitivity to exemestane or its excipients; and

(14) be willing to use a barrier method of contraception (condom, sponge, or diaphragm with spermicidal jelly) during the entire study period, unless the woman subject had a tubal ligation or her sexual partner had a vasectomy.

The woman subjects participated in the study for 5 months (156 days) divided into 4 periods:

(1) a screen period of up to 36 days (−1 to −36 days);

(2)1 month control menstrual cycle (−28 to −36 days);

(3) 2 months of treatment with either 25 mg exemestane (N=25) or 100 mg exemestane (N=28) orally per day (56 days); and

(4) 1 month of follow-up (28 days).

The primary efficacy endpoint was an improvement in dysmenorrhea, dyspareunia and pelvic pain after two months of treatment. These symptoms were evaluated using the Biberoglu and Behrman scale (using scores 0 to 3, wherein 0 represented no discomfort, 1 represented mild pain, 2 represented moderate pain and 3 represented severe pain). Dysmenorrhea, dyspareunia and pelvic pain were assessed using the ESS Scale. The criteria used to assign a score to each of the pain symptoms under the ESS Scale were as described below.

For dysmenorrhea, the criteria for a score of 1 (mild) were minimal loss of work efficiency; slight interference with usual activities of daily living; and some to occasional use of nonnarcotic analgesics or antiprostaglandin drugs. The criteria for a score of 2 (moderate) were noticeable interference with usual activities of daily living; and regular use of nonnarcotic analgesics or antiprostaglandin drugs. To obtain a score of 3 (severe), the criteria were extreme interference with usual activities of daily living; inability to function normally; and requiring narcotic analgesics.

For pelvic pain, the criteria for a score of 1 (mild) were occasional pelvic discomfort or premenstrual pain and occasional use of nonnarcotic analgesics or antiprostaglandin drugs. The criteria for a score of 2 (moderate) were noticeable discomfort during most of the menstrual cycle; and regular use of nonnarcotic analgesics or antiprostaglandin drugs. To obtain a score of 3 (severe) in pelvic pain, the criterion was constant pelvic pain requiring use of strong analgesics.

For dyspareunia, the criteria for a score of 1 (mild), 2 (moderate) and 3 (severe) were tolerated discomfort; painful to the point of interruption of intercourse; and avoidance of intercourse because of pain, respectively.

Dysmenorrhea, dyspareunia and pelvic pain were evaluated at screen, start of the first exemestane treatment cycle (baseline; visit 6), end of the first exemestane treatment cycle (4 weeks; visit 10), end of the second exemestane treatment cycle (8 weeks; end of treatment; visit 14), and end of the follow-up cycle (visit 18). A positive response was defined as an improvement, i.e. a reduction in points in the total score, from baseline of

(a) at least 2 points in the total score if 2 of dysmenorrhea, dyspareunia and pelvic pain were present at baseline; or

(b) at least 3 points in the total score if dysmenorrhea, dyspareunia and pelvic pain were all present at baseline.

Exemestane given at 25 or 100 mg orally per day produced a moderate improvement in endometriosis symptoms (see Tables 1-4). Overall, both 25 mg and 100 mg of exemestane were well tolerated by-the 54 woman subjects who received at least one dose. No noteworthy changes were found over the study period in the hematology, plasma chemistry or urinalysis laboratory assays in either treatment group. Neither blood pressure nor body weight changed significantly in either treatment group over the study period.

TABLE 1
Endometrial Symptoms: ESS Composite Improvement from
Baseline in Randomized Patients Treated with Exemestane

| | ESS Composite | Exemestane | | Exemestane | |
|---|---|---|---|---|---|
| | Improvement from | 25 mg N = 26 | | 100 mg N = 28 | |
| Study Period | Visit 6 (Baseline)⁺ | n | % | n | % |
| Visit 10 | Yes | 11 | 44.0 | 14 | 53.8 |
| (4 weeks of | No | 14 | 56.0 | 12 | 46.2 |
| exemestane) | Total Reported | 24 | | 26 | |
| Visit 14 | Yes | 8 | 33.3 | 13 | 56.5 |
| (8 weeks of | No | 16 | 66.7 | 10 | 43.5 |
| exemestane) | Total Reported | 24 | | 23 | |
| Visit 18 | Yes | 4 | 16.0 | 11 | 50.0 |
| (follow-up) | No | 21 | 84.0 | 11 | 50.0 |
| | Total Reported | 25 | | 22 | |

% = (n/Total Reported within period) × 100
⁺An improvement from the start of treatment (Visit 6) is defined as at least a 3-point decrease if all 3 symptoms were evaluated at the start of treatment (Visit 6) and at the respective time period or at least a 2-point decrease if only 2 symptoms were evaluated (a patient could enter the study without dyspareunia)

TABLE 2
Response in Individual ESS Components

| | Improvement over | Exemestane | | Exemestane | |
|---|---|---|---|---|---|
| | Visit 6 (Baseline, | 25 mg N = 26 | | 100 mg N = 28 | |
| Study Period | Start of Treatment)⁺ | n | % | n | % |
| Dysmenorrhea | | | | | |
| Visit 10 | Yes | 14 | 56.0 | 16 | 61.5 |
| (4 weeks of | No | 11 | 44.0 | 10 | 38.5 |
| exemestane) | Total Reported | 25 | | 26 | |
| Visit 14 | Yes | 14 | 58.3 | 14 | 60.9 |
| (8 weeks of | No | 10 | 41.7 | 9 | 39.1 |
| exemestane) | Total Reported | 24 | | 23 | |
| Visit 18 | Yes | 9 | 36.0 | 11 | 50.0 |
| (follow-up) | No | 16 | 64.0 | 11 | 50.0 |
| | Total Reported | 25 | | 22 | |
| Dyspareunia⁺⁺ | | | | | |
| Visit 10 | Yes | 11 | 64.7 | 11 | 61.1 |
| (4 weeks of | No | 6 | 35.3 | 7 | 38.9 |
| exemestane) | Total Reported | 17 | | 18 | |
| Visit 14 | Yes | 13 | 68.4 | 9 | 52.9 |
| (8 weeks of | No | 6 | 31.6 | 8 | 47.1 |
| exemestane) | Total Reported | 19 | | 17 | |
| | NA | 5 | | 6 | |
| Visit 18 | Yes | 12 | 70.6 | 7 | 50.0 |
| (follow-up) | No | 5 | 29.4 | 7 | 50.0 |
| | Total Reported | 17 | | 14 | |
| | NA | 8 | | 8 | |
| Pelvic Pain | | | | | |
| Visit 10 | Yes | 14 | 56.0 | 18 | 69.2 |
| (4 weeks of | No | 11 | 44.0 | 8 | 30.8 |
| exemestane) | Total Reported | 25 | | 26 | |
| Visit 14 | Yes | 14 | 58.3 | 16 | 69.6 |
| (8 weeks of | No | 10 | 41.7 | 7 | 30.4 |
| exemestane) | Total Reported | 24 | | 23 | |
| Visit 18 | Yes | 8 | 32.0 | 15 | 68.2 |
| (follow-up) | No | 17 | 68.0 | 7 | 31.8 |
| | Total Reported | 25 | | 22 | |

⁺Improvement is defined as at least a decrease of 1 point from the start of treatment (visit 6).
⁺⁺For dyspareunia, if the value at either Visit 6 or the respective time point is NA, then the value for improvement is NA

TABLE 3
Endometrial Symptoms: Change of ESS Scale
Composite Score from Baseline (Visit 6)

| | ESS Scale, | Exemestane | Exemestane |
|---|---|---|---|
| | Composite Score, | 25 mg | 100 mg |
| Study Period | Change from Visit 6⁺⁺ | N = 26 | N = 28 |
| Visit 10 (4 | Total Reported | 17 | 18 |
| weeks of | Visit 6 (Treatment Start) | 5.0588 | 5.9444 |
| exemestane) | Mean (SD)⁺⁺⁺ | (1.2485) | (1.6968) |
| | Visit 10 Mean (SD) | −2.000 | −2.5000 |
| | | (2.0917) | (2.0364) |
| | Visit 10 Min-Max | −5.000- | −6.000- |
| | | 2.000 | 1.000 |
| | Within Treatment | 0.002* | <0.001* |
| | test (p-value)⁺ | | |
| Visit 14 (8 | Total Reported | 19 | 17 |
| weeks of | Visit 6 (Treatment Start) | 5.1579 | 5.5882 |
| exemestane) | Mean (SD)⁺⁺⁺ | (1.2140) | (1.5835) |
| | Visit 14 Mean (SD) | −2.000 | −2.4118 |
| | | (1.8257) | (1.9059) |
| | Visit 14 Min-Max | −6.000- | −6.000- |
| | | 1.000 | 0.000 |
| | Within treatment test | <0.001* | <0.001* |
| | (p-value)⁺ | | |
| Visit 18 | Total Reported | 17 | 14 |
| (follow-up) | Visit 6 (Treatment Start) | 5.0588 | 5.5714 |
| | Mean (SD)⁺⁺⁺ | (1.2485) | (1.6508) |
| | Visit 18 Mean (SD) | −2.000 | −1.4286 |
| | | (1.8028) | (1.7852) |
| | Visit 18 Min-Max | −6.000- | −5.000- |
| | | 0.000 | 2.000 |
| | Within treatment test | <0.001* | 0.016* |
| | (p-value)⁺ | | |

⁺Statistical Test(s) used in the table include KRUSKAL-WALLIS and WILCOXON SIGNED RANK
⁺⁺Composite score based on patients with 3 endpoints evaluated at both visits
⁺⁺⁺Summary statistics of Visit 6/Trt Start values for patients having non missing values at both Reference Period and Study Period.
*P-value ≦ 0.05 for the overall treatment test.
*P-value ≦ 0.05 for the within treatment test

TABLE 4
Change from Baseline to Visit 10 and Visit 14 for Dysmenorrhea,
Dyspareunia and Pelvic Pain
Study Visit

| Visit 6 | | | Visit 10 | | Visit 14 | |
|---|---|---|---|---|---|---|
| (Baseline) | | | (4 weeks of exemestane) | | (8 weeks of exemestane) | |
| Exemestane | | | Exemestane | | Exemestane | |
| | 25 mg | 100 mg | 25 mg | 100 mg | 25 mg | 100 mg |
| Dysmenorrhea | | | | | | |
| Mean ± SD | 2.0 ± 0.6 | 2.1 ± 0.7 | −0.9 ± 0.8 | −1.0 ± 1.3 | −0.8 ± 0.6 | −0.8 ± 0.7 |
| Range | | | −2-0 | −3-0 | −2-0 | −2-0 |
| N | 26 | 28 | 17 | 18 | 19 | 17 |
| Dyspareunia | | | | | | |
| Mean ± SD | 1.6 ± 0.7 | 1.9 ± 1.0 | −0.9 ± 0.8 | −1.0 ± 1.3 | −0.8 ± 0.6 | −0.8 ± 0.8 |
| Range | | | −2-0 | −3-1 | −2-0 | −2-0 |
| N | 21 | 21 | 17 | 18 | 19 | 17 |
| Pelvic Pain | | | | | | |
| Mean ± SD | 1.8 ± 0.5 | 2.1 ± 0.5 | −0.6 ± 0.9 | −0.8 ± 0.7 | −0.6 ± 0.9 | −0.8 ± 0.8 |
| Range | | | −2-2 | −2-1 | −2-1 | −2-1 |
| N | 26 | 28 | 25 | 26 | 24 | 23 |

We claim:

1. A method for preventing and/or treating an estrogen-dependent disorder selected from endometriosis, uterine fibroids, dysfunctional uterine bleeding, endometrial hyperplasia, polycystic ovarian syndrome, fibrocystic breast disease and fibrocystic mastopathy in a female mammal in need of the prevention, comprising administering an effective amount of exemestane to the mammal.

2. A method according to claim 1, further comprising administering at least one additional therapeutic agent to the mammal.

3. A method according to claim 2, wherein the at least one additional therapeutic agent is selected from the group consisting of danazol, a COX-2 inhibitor, a non-steroidal anti-inflammatory compound (NSAID), a retinoid compound, a matrix metallo-protease inhibitor, an anti-estrogen, GnRH agonist or antagonist, a selective progestin receptor modulator (SPRM) and an angiogenesis inhibitor, or a mixture thereof.

4. A method according to claim 2, wherein the at least one additional therapeutic agent comprises from 2 to 4 subtances selected from the group consisting of danazol, a COX-2 inhibitor, a non-steroidal anti-inflammatory compound (NSAID), a retinoid compound, a matrix metallo-protease inhibitor, an anti-estrogen, GnRH agonist or antagonist, a selective progestin receptor modulator (SPRM) and an angiogenesis inhibitor.

5. A method according to claim 3, wherein the at least one additional therapeutic agent is anti-estrogen, which is a SERM devoid of uterotrophic activity.

6. A method according to claim 5, wherein the SERM is selected from the group consisting of tamoxifen, toremifene, arzoxifene, idoxifene, EM 800, fulvestrant and droloxifene.

7. A method according to claim 3, wherein the at least one additional therapeutic agent is a GnRH agonist selected from the group consisting of leuprorelin, dislorelin, triptorelin, buserelin, nafarelin, goserelin, avorelin, histerelin, compound PTL 03301, compound AN 207, compound TX 397, compound AN 201 and compound SPD 424, or a pharmaceutically acceptable salt thereof.

8. A method according to claim 7, wherein the GnRH agonist is selected from the group consisting of triptorelin, goserelin and leuprorelin or a pharmaceutically acceptable salt thereof.

9. A method according to claim 8, wherein the GnRH agonist is triptorelin pamoate.

10. A method according to claim 3, wherein the GnRH antagonist is selected from the group consisting of cetrorelix, abarelix, ramorelix, teverelix, ganirelix, compounds A 75998 and A 84861, compound PM-OV-92; GnRH immunogen, compound D 26344, compound T 98475 and compound MI 1544, or a pharmaceutically acceptable salt thereof.

11. A method according to claim 10, wherein the GnRH antagonist is abarelix or a pharmaceutically acceptable salt thereof.

12. A method according to claim 3, wherein the at least one additional therapeutic agent is a SPRM, which is dienogest or a pharmaceutically acceptable salt thereof.

13. A method according to claim 3, wherein the at least one additional therapeutic agent is a COX-2 inhibitor selected from the group consisting of: 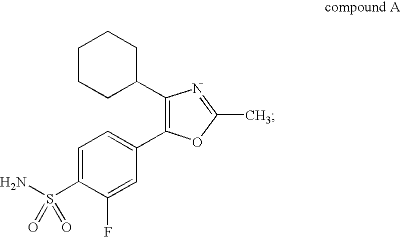
5-chloro-3-(4-(methylsulfonyl)phenyl)-2-(methyl-5-pyridinyl)pyridine;
2-(3,5-difluorophenyl)-3-4(methylsulfonyl)phenyl)-2-cyclopenten-1-one; 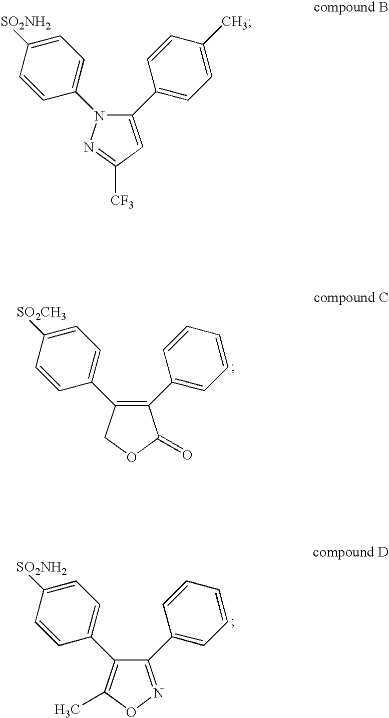
N-[[4-(5-methyl-3-phenylisoxazol-4-yl]phenyl]sulfonyl]propanamide; 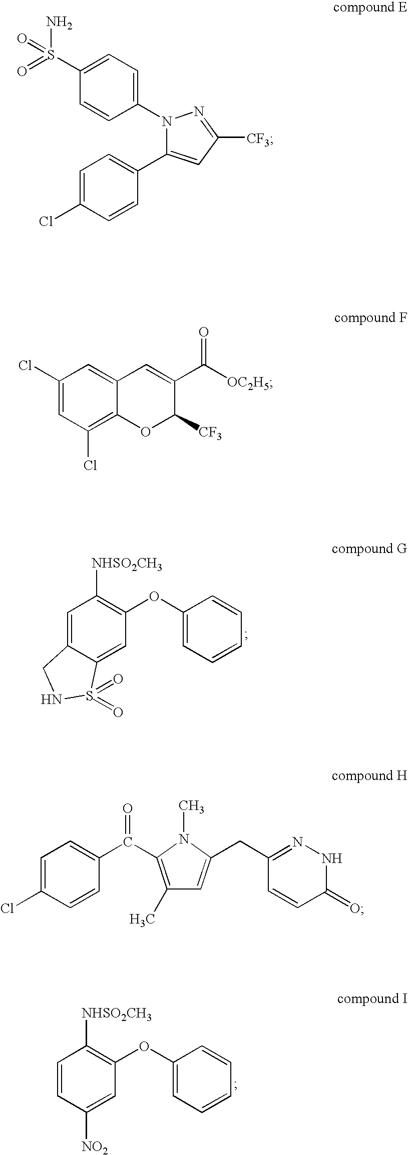 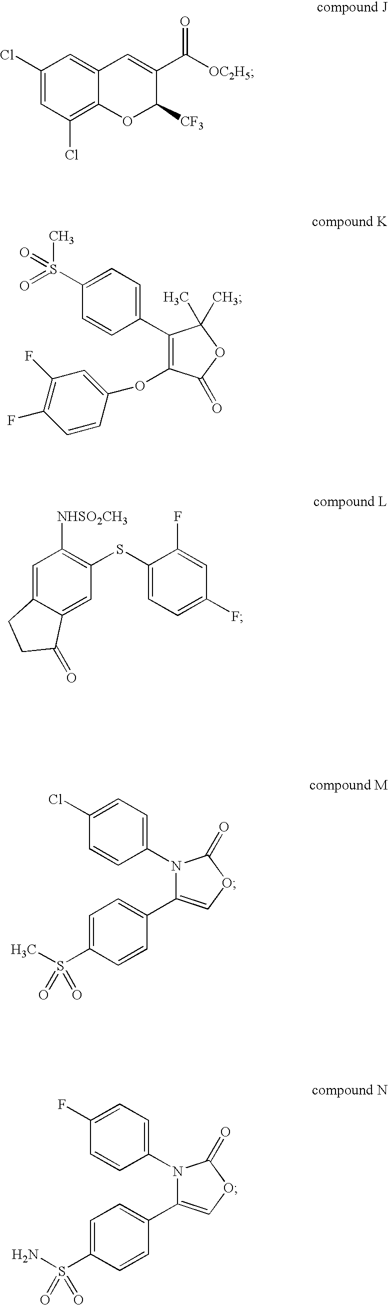 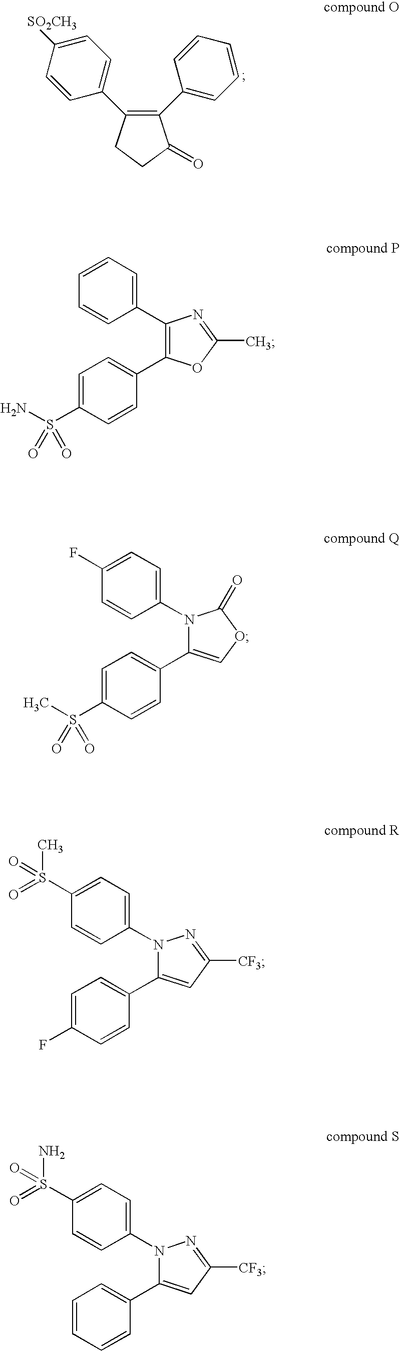 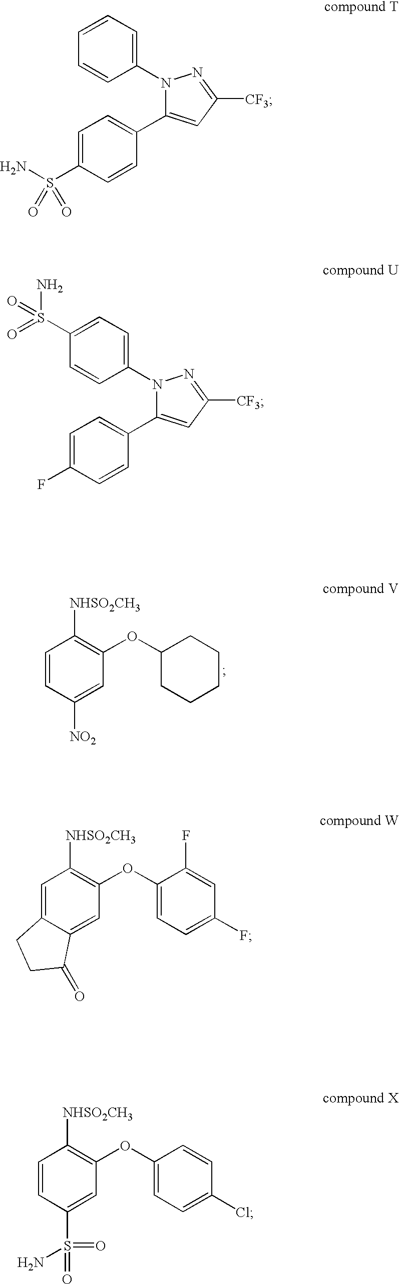 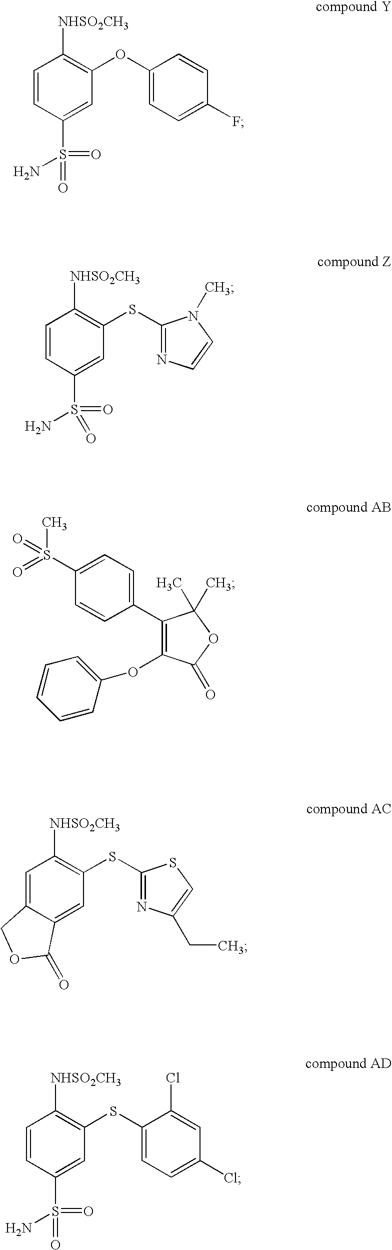 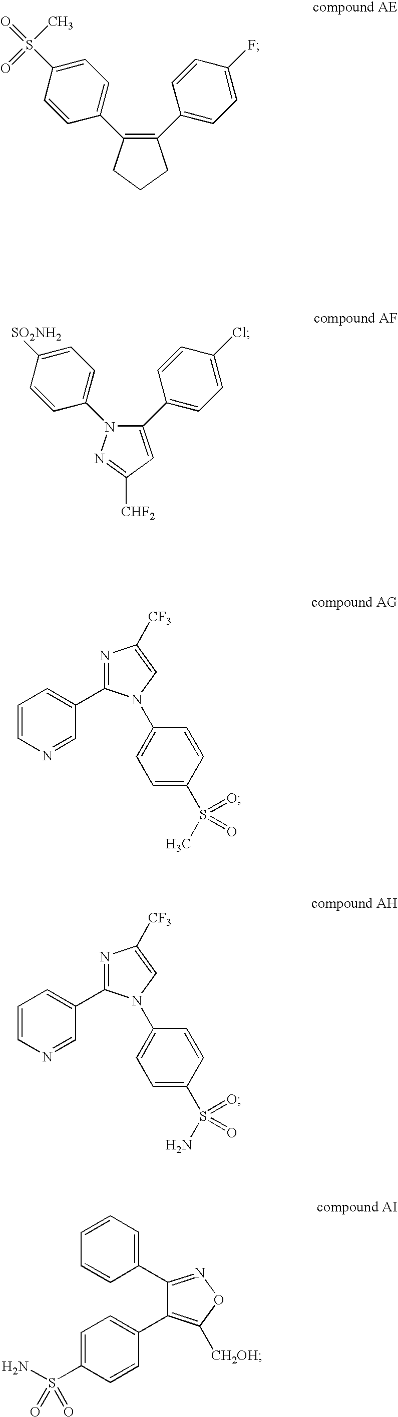 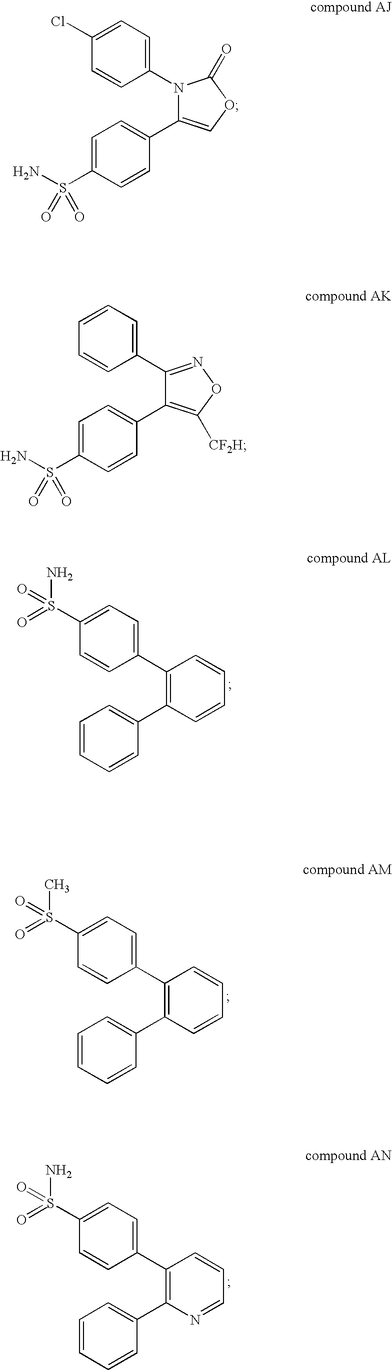 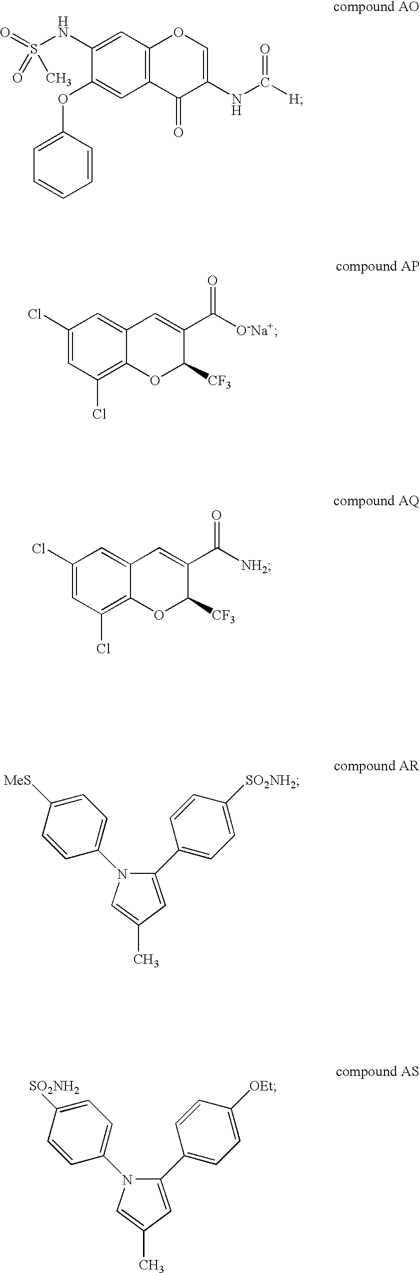
compound T 614; darbufelone; compound L745337; celecoxib; compound CT3; rofecoxib; compound L783003; compound JT3522; compound 754; parecoxib; compound S2474; compound LAS 33815; valdecoxib; and compound MK 663.

14. A method according to claim 3, wherein the at least one additional therapeutic agent is a COX-2 inhibitor selected from celecoxib, rofecoxib, parecoxib and valdecoxib.

15. A method according to claim 14, wherein the COX-2 inhibitor is celecoxib.

16. A method according to claim 3, wherein the at least one additional therapeutic agent is a NSAID selected from the group consisting of acetyl salicylic acid, indometacin, sulindac, phenylbutazone, diclofenac, fentiazac, ketorolac, piroxicam, tenoxicam, mecoxicam, meloxicam, cinnoxicam, ibufenac, ibuprofen, naproxen, ketoprofen, nabumetone, niflumic acid and nimesulide, or a pharmaceutically acceptable salt thereof.

17. A method according to claim 16, wherein the NSAID is selected from the group consisting of diclofenac, piroxicam, tenoxicam, mecoxicam, meloxicam, ibufenac, ibuprofen, naproxen and ketoprofen, or a pharmaceutically acceptable salt thereof.

18. A method according to claim 3, wherein the at least one additional therapeutic agent is a retinoid compound selected from the group consisting of Accutane; Adapalene; AGN-193174; AGN-193676; AGN-193836; AGN-193109; AR-623; BMS-181162; CD-437; ER-34617; Etrinate; Fenretinide; Ligand LGD-1550; lexacalcitol; MX-781; mofarotene; MDI-101; MDI-301; MDI-403; Motretinide; 4-(2-[5-(4-methyl-7-ethylbenzofuran-2-yl)pyrrolyl])benzoic acid; N-[4-[2-thyl-1-(1H-imidazol-1-yl)butyl]phenyl]-2-benzothiazolamine; Soriatane; SR-11262; Tocoretinate; Advanced Polymer Systems trans-retinoic acid; UAB-8; Tazorac; TopiCare; TAC-101; and Vesanoid.

19. A method according to claim 3, wherein the at least one additional therapeutic agent is a metallo-protease inhibitor selected from the group consisting of:
1-cyclopropyl-N-hydroxy-4-[[4-[4-(trifluoromethoxy)phenoxy]phenyl]sulfonyl]-4-piperidinecarboxamide monohydrochloride;
N-hydroxy-1-(phenylmethyl)-4-[[4-[4-(trifluoromethoxy)phenoxy]-1-piperidinyl]sulfonyl]-4-piperidinecarboxamide monohydrochloride;
N-hydroxy-1-(pyridinylmethyl)-4-[[4-[4-(trifluoromethyl)phenoxy]phenyl]sulfonyl]-4-piperidinecarboxamide dihydrochloride;
N-hydroxy-2,3-dimethoxy-6-[[4-[4-(trifluoromethyl)phenoxy]-1-piperidinyl]sulfonyl]-benzamide;
N-hydroxy-1-(4-pyridinylmethyl)-4-[[4-[4-(trifluoromethyl)phenoxy]phenyl]sulfonyl]-4-piperidinecarboxamide dihydrochloride;
N-hydroxy-1-(3-pyridinylmethyl)-4-[[4-[4-(trifluoromethyl)phenoxy]phenyl]sulfonyl]-4-piperidinecarboxamide dihydrochloride;
N-hydroxy-1-(2-pyridinylmethyl)-4-[[4-[4-(trifluoromethyl)phenoxy]phenyl]sulfonyl]-4-piperidinecarboxamide monohydrochloride;
BB-2516 (marimastat), N4-[2,2-dimethyl-1-[(methylamino)carbonyl]-propyl]-N1,2-dihydroxy-3-(2-methylpropyl)-, [2S-[N4(R*), 2R*, 3S*]]-);
BMS 275291;
Bay-12-9566 (tanomastat), 4-[(4′-chloro[1,1-diphenyl]-4-yl)oxy]-2-[(phenylthio) methyl]butanoic acid;
AG-3340, N-hydroxy-2,2′-dimethyl-4-[[4-(4-pyridinyloxy)phenyl]sulfonyl]-3-thiomorpholine-carboxamide;
CMT-3 (metastat), 6-demethyl-6-deoxy-4-dedimethylaminotetracycline, batimastat (BB-94); and
D-2163,2-[1S-([(2R,S)-acetylmercapto-5-phthalimido]pentanoyl-L-leucyl)amino-3-methylbutyl]imidazole.

20. A method according to claim 3, wherein the at least one additional therapeutic agent is an angiogenesis inhibitor selected from the group consisting of an αvβ3 integrin inhibitor, a protein kinase inhibitor, angiostatin, platelet factor 4 (endostatin), a VEGF inhibitor and thalidomide.

21. A method according to claim 20, wherein the angiogenesis inhibitor is thalidomide.

22. A method according to claim 20, wherein the αvβ3 integrin inhibitor is selected from the group consisting of:
Vitaxin antibody (Ixsys); Merck KgaA EMD-121974, cyclo[RGDF-N(Me)V-];
(10S)-10,11-dihydro-3-[3-(2-pyridinylamino propoxy]-5H-dibenzo[a,d]cycloheptene-10-acetic acid;
(2S)-7-[[(1H-benzimidazol-2-ylmethyl)methylamino]carbonyl]-2,3,4,5-tetrahydro-4-methyl-3-oxo-1H-1,4-benzodiazepine-2-acetic acid;
(2S)-2,3,4,5-tetrahydro-4-methyl-7-[[[(5-methyl-1H-imidazo[4,5-b]pyridin-2-yl]methyl]amino]carbonyl]-3-oxo-1H-1,4-benzodiazepine-2-acetic acid;
(bR)-b-[[[(3R)-2-oxo-3-[2-(5,6,7,8-tetrahydro-[1,8]-naphthyridin-2-yl)ethyl]1-1-pyrrolidinyl]acetyl]amino]-d-(1H indol-3-yl)pentanoic acid; and
(3R)-N-[3-hydroxy-5-[(1,4,5,6-tetrahydro-5-hydroxy-2-pyrimidinyl)amino]benzoyl]-glycyl-3-(3-bromo-5-chloro-2-hydroxyphenyl)-b-alanine (compound SD 7784).

23. A method according to claim 20, wherein the protein kinase inhibitor is selected from compound SU6668 (3-[4-(2-carboxyethyl-3,5-dimethylpyrrol-2-yl)methylidenyl]-2-indolinone), and compound SU5416 (3-[(2,4-dimethylpyrrol-5-yl)methylidenyl]-2-indolinone).

24. A method according to claim 20, wherein the VEGF inhibitor is selected from the group consisting of compound SU 6668, compound SU 5416, rhuMAbVEGF and compound DC 101.

25. A method according to claim 1, wherein the mammal is a human.

26. A pharmaceutical composition for preventing and/or treating an estrogen-dependent disorder, said preparation comprising exemestane and at least one additional therapeutic agent.

27. The composition according to claim 26, wherein the at least one additional therapeutic agent is selected from the group consisting of danazol, a COX-2 inhibitor, a non-steroidal anti-inflammatory compound (NSAID), a retinoid compound, a matrix metallo-protease inhibitor, an anti-estrogen, GnRH agonist or antagonist, a selective progestin receptor modulator (SPRM) and an angiogenesis inhibitor, or a mixture thereof.

28. A method for treating infertility in a female mammal in need of the treatment, comprising administering an infertility-treating effective amount of exemestane to the mammal.

29. A method according to claim 28, wherein the infertility is an estrogen-dependent disorder.

30. A method according to claim 28, wherein the infertility is caused by or associated with endometriosis.

31. A method according to claim 28, wherein the infertility is caused by or associated with polycystic ovarian syndrome.

32. A method according to claim 28, wherein the infertility is anovulatory infertility.

33. A method according to claim 28, wherein the mammal has hypogonadotropic hypogonadism.

34. A method according to claim 28, wherein the mammal has a menstrual cycle disorder.

35. A method according to claim 28, wherein the mammal is a human.

36. A method according to claim 28, wherein exemestane is administered in day 5 to day 7 of a menstrual cycle of the mammal and then stopped.

37. A method according to claim 28, wherein exemestane is administered throughout a menstrual cycle of the mammal and then stopped.

38. A method according to claim 28, wherein the effective amount is a therapeutically effective follicular stimulating amount.

39. A method according to claim 28, wherein the mammal is a candidate for an assisted reproduction technique.

40. A method for inducing ovarian follicular stimulation in a female mammal in need of the induction, comprising administering a therapeutically effective follicular stimulating and/or inhibitory amount of exemestane to the mammal, followed by a stoppage of the exemestane administration, wherein the stoppage provides rebound hyperstimulation of the ovaries.

41. A method according to claim 40, wherein the mammal is a human.
